(19) United States
(12) Patent Application Publication
(10) Pub. No.: US 2015/0047649 A1
(43) Pub. Date: Feb. 19, 2015

(54) THERMAL AND HYDRATION THERAPY MASK WITH EASILY REMOVABLE THERMAL PADS

(71) Applicant: Suzanne Paulson, Temecula, CA (US)

(72) Inventor: Suzanne Paulson, Temecula, CA (US)

(21) Appl. No.: 13/969,412

(22) Filed: Aug. 16, 2013

(51) Int. Cl. A61F 9/04; A61H 35/02; A61F 7/08

(52) U.S. Cl. CPC A61F 9/04; A61F 7/086; A61H 35/02; A61F 2007/0004
USPC 128/858

(57) ABSTRACT

An eyewear apparatus which maintains a sealed cavity in front of a user's eye area when engaged to the face in an as-worn position. The device features a mask configured to contain one or both of thermal and moisture pads. The projecting edge portion surrounding said mask provides a seal to maintain the cavity between the device and the user's face to contain the temperature and humidity differential. Plugs are designed to allow the user to replace or resupply either or both of the thermal and moisture pads without removing the mask from the wearer.

### FIELD OF THE INVENTION

The disclosed device and method, relate to an apparatus enabling a system and method for eye hydration therapy. More particularly, the system provides enhanced hydration therapy therethrough the employment of removably engageable heated and moisturized inserts which when engaged within a face mask, communicate humidity and hydration therapy to the user's eyes. The device in an as-used engagement over the user's face to form a sealed cavity in communication with the user's eyes, is employable to relieve the symptoms of eye disorders such as Dry Eye, Blepharitis and Meibomian Gland Dysfunction.

### BACKGROUND OF THE INVENTION

Diseases and conditions relating to dry eye, are widespread in the United States where as many as 20-25% of the population suffers from one or more eye disorders relating to dry eyes.

Of the approximately 60 million Americans who suffer from dry eye conditions, more than 10 million Americans have been diagnosed with severe dry eye conditions such as Dry Eye Syndrome or Chronic Dry Eye Disease (CDED). Both of these conditions are conventionally known as Keratoconjunctivitis sicca and both are extremely hard to treat effectively. CDED stems from inflammation in the eyes and tear-producing glands. This inflammation causes a decrease in the ability to produce and communicate natural tears to the eyes. The resulting lack of tears causes a breakdown in the patient's body of its natural ability to clean, protect and moisten the eyes.

Further, approximately 75% of the 60 million people suffering from dry eye symptoms experience Evaporative Dry Eye, which is caused by a lack of natural oil in the content of produced tears. The remainder of Dry Eye is categorized as aqueous deficient Dry Eye. In patients with aqueous deficient Dry Eye, there is a decrease in one or both of the quality and quantity of the tears produced naturally.

CDED as noted above, is often a result of normal aging of the eye, however such symptoms of dry, irritated, tired eyes can occur in patients at any age. Symptoms of a CDED condition are experienced by approximately 75% of men and women over the age of 65, but are mostly prevalent among women who are postmenopausal or pregnant. Approximately four million of those Americans, experience CDED as a symptom of Sjogren's syndrome, a chronic, slowly progressive autoimmune disease characterized by dryness of the eyes and mouth and recurrent salivary enlargement, although other diseases, such as rheumatoid arthritis, lupus, scleroderma, and thyroid disease, can also cause CDED.

Health, environmental, and lifestyle conditions, can also trigger CDED. In the United States for example, many of the 50 million people suffering from allergies, the 38 million Americans who wear contact lenses, have a greater risk of developing CDED.

Other circumstances which cause can significantly dry, irritate, and fatigue the eye of both children and especially adults, include, computer use, living or working in environments where humidity is between 5% to 15% and thus very low, flying in an airplane which maintain low humidity as a rule, ingesting or using certain medications, eye surgery, and certain medical conditions, such as blepharitis (inflammation of the eyelids). As can be seen, CDED affects a major portion of the population in one form or another, either in an ongoing or temporary fashion depending on the patient and the venue or environment.

Some early symptoms suffered by patients with CDED include: 1) temporary ocular burning sensations; 2) a persistent, painful gritty sensation in the eyes; 3) an inability to cry under emotional stress; 4) decreased tolerance of contact lens use; and 5) in extreme cases, unusual sensitivity to light, severe eye pain, or diminished vision. The symptoms of CDED often increase from morning to night, intensify from periodic to persistent pain if untreated.

As the American population ages, and with the increasing prevalence of pollution, chemical allergens, eye taxing computer and cell phone screens, and air travel and the like, CDED diagnoses will continue to rise. Concurrently, the need for improved methods and devices for the effective treatment of CDED will escalate.

In patients suffering from CDED, in order to effectively hydrate the eyes and reduce eye inflammation, sufficient heat must be communicated to the eyes of the patient to express oils from the meibomian glands. However, this communication of heat must be accomplished without burning or irritating the skin and internal surfaces of the eyelids and eye-surrounding skin tissue.

A conventional treatment employed and recommended by physicians for decades, is the use of moistened hot wash towel. The hot and moist towel is placed in contact with and over the eyelids, for a period of up to 10 minutes and sometimes more, to help moisten the eyes and relieve symptoms of CDED.

Such towels and moist cloths however, being exposed to surround air which is cooler than the towel itself, tend to cool to room temperature within three minutes as the heat in the towel communicates to the cooler atmosphere. As a consequence, the patient, or the user treating a patient, must constantly disrupt their therapy to reheat and reapply the washcloth.

In addition, temperature and humidity regulation with this moist towel method is difficult. This is because the moisture communicated is very dependant on the selected cloth and the temperature of the water with which it is saturated. Less absorbent fabric may hold less moisture, and some fabrics are prone to more easily communicate the moisture with the surrounding atmosphere, and thereby cause a faster decrease in both temperature and fluid content.

Additionally, the employment and use of moistened towels and cloths is at best, cumbersome. This is because such washcloths being exposed to pathogens present on a user's skin, and floating in the air, must be thoroughly cleaned to reduce the risk of subsequent communication of such pathogens or bacteria to the eye of a patient resulting in eye infection.

Still further, moist towels such as terrycloth or other fabrics, easily drip, especially if over-saturated, and require the user to remain relatively still and recline during use as movement increases such drip. Even absent such movement, the slow spread of multiple drips down the patient's face and neck can be extremely irritating during a treatment session which is supposed to be calming.

Doctors may also prescribe heat and moisture treatment to the eyes for related surgery rehabilitation or relaxation. A similar, but inverse, treatment is also prescribed to mitigate persistent headaches and migraines through a cold compress intended to communicate cold temperatures to the eyes. As can be discerned, the anticipation of a cold towel and cold drips and running of water on a patient's face is not always eagerly received.

Prior art has yielded a few therapeutic masks which are adapted to replace the wash cloth or cold compress in communicating heat or cold to the user's eyes. Some such devices also attempt to segregate heat or cold to the eye area through embedded or removable enclosed pods which can be moistened and heated.

One goggle apparatus, for heating and hydrating the eyes, has been described per U.S. Pat. No. 7,231,922. With this device, the user places a removable heated pad in each goggle socket and places the device over and around their head, so that the pads cover their eyes for the prescribed period. If the duration of heat, or cold, and hydration which can be provided by the removable pads is less than the prescribed treatment time, however, the user must remove the apparatus to reinsert new heated pods, and thus disrupt the stored temperature differential and heated humid atmosphere within the goggles.

In addition, although a goggle type device may communicate the heat or cold to the most affected area in the tissue directly surrounding the eyes, it is also considered important to apply therapy to the entire ocular facial area. Such therapy ensures that surrounding dry tissue does not draw moisture or heat away from the affected area during treatment or once treatment ceases. Further, such surrounding tissue temperature communication is important to increase the therapeutic extent the for facial surgery recovery, and the treatment of migraines.

As such, there is an unmet need for a thermal and hydrating system and component for the eyes and surrounding tissue of a patient, which allows the user to replenish the heat and source of moisture quickly and easily without removing the apparatus. Such a device should be configured to treat the entire ocular facial area or a portion thereof as needed. Such a device should be configured to communicate heat or cold at will, and thereby even be able to provide progressive hot and cold therapies to a patient, without removal of the main component of the device providing the sealed chamber adjacent the eyes and facial surface of the patient. Still further, such a device should, when worn by a patient, allow an attendant to easily resupply the heat or cold source and/or moisture to the treatment cavity, without the need for the patient to move and/or remove the device forming the treatment chamber communicating temperature and/or humidity to the tissue of the eyes and surrounding area of the patient.

The device and method described herein, accordingly comprises the features of such a device, in the construction, use, combination of elements, and arrangement of parts, which will be exemplified by the constructions hereinafter set forth, and the scope of the invention will be indicated in the claims.

### SUMMARY OF THE INVENTION

The disclosed device and method, employs a face mask style component, configured to form a treatment cavity adjacent the eyes and the surrounding tissue of the eyes of a user or patient. This treatment chamber, when sealed from the outside atmosphere, communicates a hydrated atmosphere to the area of the eyes and surrounding skin of the patient, providing a means for maintaining the eyes in a controlled atmosphere which is in a hydrated and heated, or cooled condition. Since the use of moist-heat is more therapeutic than dry heat, the device herein provides a much more therapeutic treatment to the wearer or user.

Maintaining the atmosphere in the treatment chamber both moist and heated, which allows the moist heat to penetrate deeply in the treated area adjacent the chamber and thereby promotes circulation. Additionally the moist heat hydrates the sensitive eye lid and surrounding skin and the entire area remains covered for the entire treatment. Further, due to the area of the treatment chamber and the provision of moist heat, the device and method herein is able to affect a much greater area of skin while also treating the lid margin.

Unlike prior art, thermal and/or moisture pads herein, providing physical contact and/or atmospheric communication, of temperature and humidity are engageable to the formed facemask to communicate temperature and moisture to the treatment cavity, through insertion into a wall of the facemask. In the favored mode where such insertion is accomplished, such is provided from exterior the endwall of the facemask, opposite the users eyes. Such engagement may be accomplished without the need for the facemask to be dismounted and can allow the user to lay prone or in a chair while the medical or other personal attend to the treatment.

Thus with the facemask in the as-used position with the sealing end of the sidewall surrounding the eye and face area of the patient, the pads and gel packs providing heat and moisture pads may all be engaged through the opposing sidewall of the facemask from the eyes, and engaged in an insertion direction toward the face of the patient.

While in another mode of the device, the thermal pads or gel packs and/or moisture pads are configured to be inserted from the inside surface, if such is desirable by users, engagement from the exterior offers more utility. Using exterior engagement, the wearer need not dismount the facemask for initial or subsequent engagement of thermal an/or moisture pads but may remain prone or sitting with the facemask attached. As noted, this is especially useful in a medical or spa setting where the user is prone and relaxing and being attended to by professionals.

Additionally, the therapeutic effects of temperature and/or humidity are not constrained to the area directly covering and surrounding the eyes, but instead may be communicated to areas adjacent the eyes and within the formed treatment chamber of the facemask. Still further, using the exterior engagement allows for sequential treatments where heat and cold are sequentially engaged to the treatment cavity.

The facemask component is formed of a sidewall having a first edge extending to a contact surface which is adapted for sealed engagement with the face. The opposite or second end of the sidewall intersects an endwall running somewhat normal to the sidewall and in a shape conforming to the arch of the user's face. The sealed engagement of the contact surface with the skin of the face forms a protective shell defining a treatment cavity between the sidewall of the facemask, the endwall, and the skin of the face of the patient.

In the favored mode where temperature and/or moisture pads are positioned within the treatment cavity from the exterior side of the endwall, apertures sized for pad insertion, for placement of heat or cold and/or moisture into the treatment cavity, are operatively positioned in the endwall of the facemask.

The sidewall of the facemask extending from the endwall, to a permiter edge adapted or configured for a sealed engagement against the face of the user, forming a sealing perimeter edge portion. The sidewall permiter at this seal, and the endwall is configured to form the treatment cavity between the endwall of the facemask and the skin of the patient, in a configuration in a shape of the ocular facial area and surrounding skin areas. Perforated walls may also be positioned behind the pad insertion apertures and within the treatment cavity to help constrain and secure the thermal pads in the device if the pads or gel packs are disengaged from the plugs engaging the apertures.

With the facemask engaged to an as-used position on the face and forming a treatment cavity surrounding and adjacent the skin of the eyes and surrounding tissue of the eyes, once the thermal pads provided by gel packs which may be heated, or chemically activated packs, and/or hydrated pad components are engaged with the apertures in the endwall, the sealing plug providing the engagement with the aperture acts to seal the treatment cavity and maintain the heat, cold and humidity within the mask, and away from the atmosphere which can quickly disperse it.

In a preferred form, the sealing plugs have a circular shape and a V-shaped cross section, and are made of a pliable material. By pinching the plug's flat V walls to compress the circular portion, the user can insert a plug into each of the complimentary apertures. Thereafter, upon release of the compression, the elastic circular portion rebounds in shape to allow the plug's perimeter to seal against the shell. Alternative sealing plugs, if constructed from a stiffer material, can screw into or plug into the apertures, or be configured to snap in and out. These and all such modes of cooperative sealed engagement of the plugs with the apertures as would occur to those skilled in the art are anticipated within the scope of this patent.

The mask and sealing plugs can be constructed from formable materials including from a group of such materials including any one or a combination of foam, rubber, silicone, latex, fiberglass, polymeric material, or any form of plastic material employable to molding the sidewall and endwall. All portions of the mask which are designed to come in direct contact with the face should be soft and flexible but durable and a soft gasket may be installed on the edge of the sidewall contacting the face. All porous materials should contain watertight covers or surfaces for easy cleaning. The mask and plug can be designed to be transparent opaque or preferably light insulating, for treating migraines.

Means for biased engagement of the sidewall against the face of the user to maintain a good seal, can be provided by straps or flexible bands. The band extends from both ends of the facemask and around the head of the user and is preferably slightly elastic so as to impart a bias to press the mask against the user's face.

In an as-worn position, with the flexible seal edge of the sidewall in contact with the user's face, and surrounding their eye area and forming a seal therewith, the product forms an interior treatment cavity in-between the endwall, sidewall, and the face. Sealing the thermal pads within the device creates an interior cavity with controlled humidity and temperature per the prescribed therapeutic remedy. The mask can also incorporate heatable gel pack covers for the endwall and/or sidewall, in a variety of fabrics, texture and shapes. The mask might utilize one large gel pack or two or more gel packs (heat source) allowing for them to have the greatest performance.

Further, one or more gel packs and fabric cover can easily be heated in a microwave, hot water, or conventional oven, cabbie (used in spas to heat towels), using steam, prior to use. Should cold therapy be desirable, gel packs can be cooled in ice, freezer, ice water, or refrigerator and then be engaged with the facemask to communicate cold to the treatment cavity.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the presently disclosed device. It is important, therefore, that the claims be regarded as including such equivalent construction and methodology insofar as they do not depart from the spirit and scope of the present invention.

It is an object of this invention to provide a therapeutic thermal mask, dimensioned to form a treatment cavity adjacent the entire eye area of the face.

It is an additional object of this invention to provide means for easily replacing thermally conductive pads or packs to communicate temperature differential within the treatment cavity of the mask, as a means to regulate the temperature and humidity, without disrupting the period of therapy.

These together with other objects and advantages which become subsequently apparent reside in the details of the construction and operation as more fully hereinafter described and claimed, reference being had to the accompanying drawings forming a part thereof, wherein like numerals refer to like parts throughout.

### BRIEF DESCRIPTION OF THE DRAWING FIGURES

In the drawing figures, which are not to scale and which are merely illustrative and wherein like reference characters denote similar elements throughout the several views:

FIG. 1 is a front perspective view of the facemask apparatus formed of an endwall having a sidewall or a perimeter edge portion projecting therefrom at an angle adapted to seal within the orbital bone around the eye area. The facemask is adapted to engage a flexible strap for securement to an as-worn position.

FIG. 2 is an overhead view of the device in an as-worn position on the face of a patient or user lying down.

FIG. 3 is a top view of the device as it would appear in the as-worn position showing the sidewall projecting around the permiter of the endwall.

FIG. 4 is a sliced view through FIG. 3, showing the perimeter features which seal the mask against the face and the placement of temperature and hydrating pads within the cavity.

FIG. 5 is a detailed sliced view of one section of the facemask and separated pinched sealing plug adjacent a complimentary aperture in the endwall.

FIG. 6 is a detailed sliced view of the device of FIG. 5, showing the plug having a sealed removable engagement with the facemask in the complimentary aperture of the endwall.

FIG. 7 depicts a mode of the device wherein the gel pack providing thermal conduction and/or the hydrating pad are engaged with the plug, and removable and replaceable as a unit.

FIG. 8 depicts the unitary plug with thermal and hydrating pads removed from the aperture of FIG. 7.

FIG. 8A shows a unitary plug and pad component as in FIG. 8, wherein both a temperature pad for hot or cold and a moisture pad, are engaged to the plug although the combination can be employed with any mode or embodiment herein.

FIG. 9 show a mode of the plug of any of the figures herein, adapted for an insertion of a supplemental heat, cooling, or hydrating component.

FIG. 10 depicts a mode of the device, with a plug which inserts from the interior side of the endwall within the treatment cavity.

### DETAILED DESCRIPTION OF THE PREFERRED EMBODIMENTS OF THE INVENTION

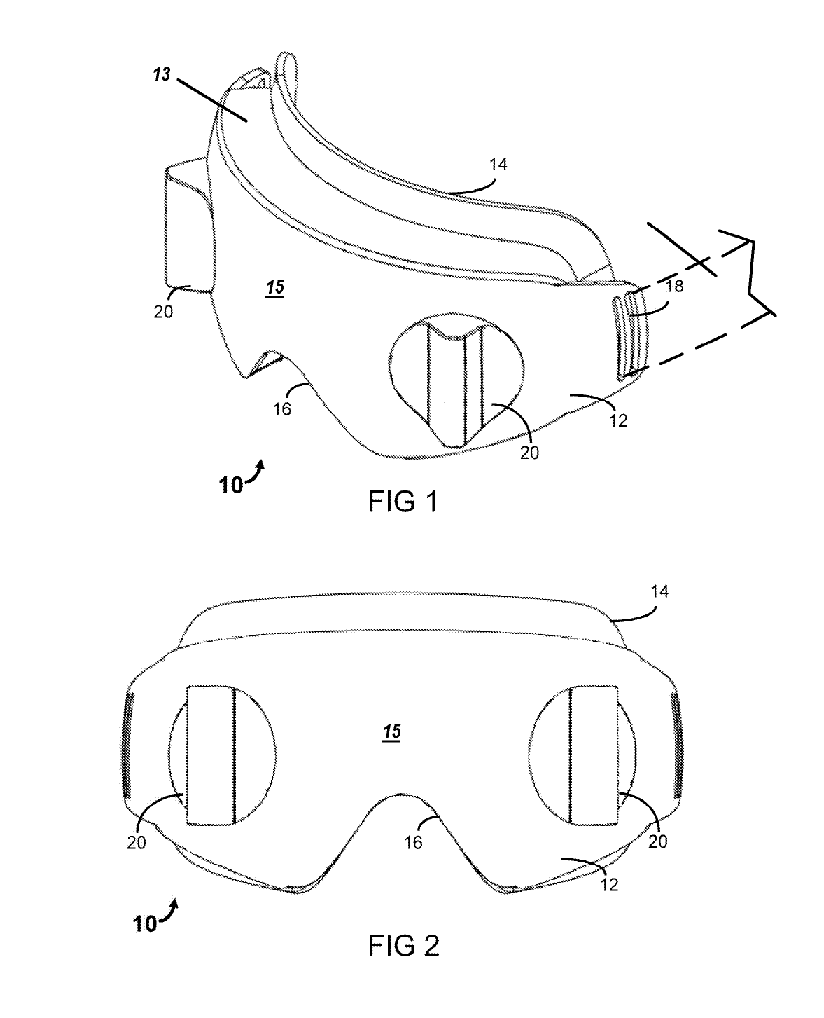

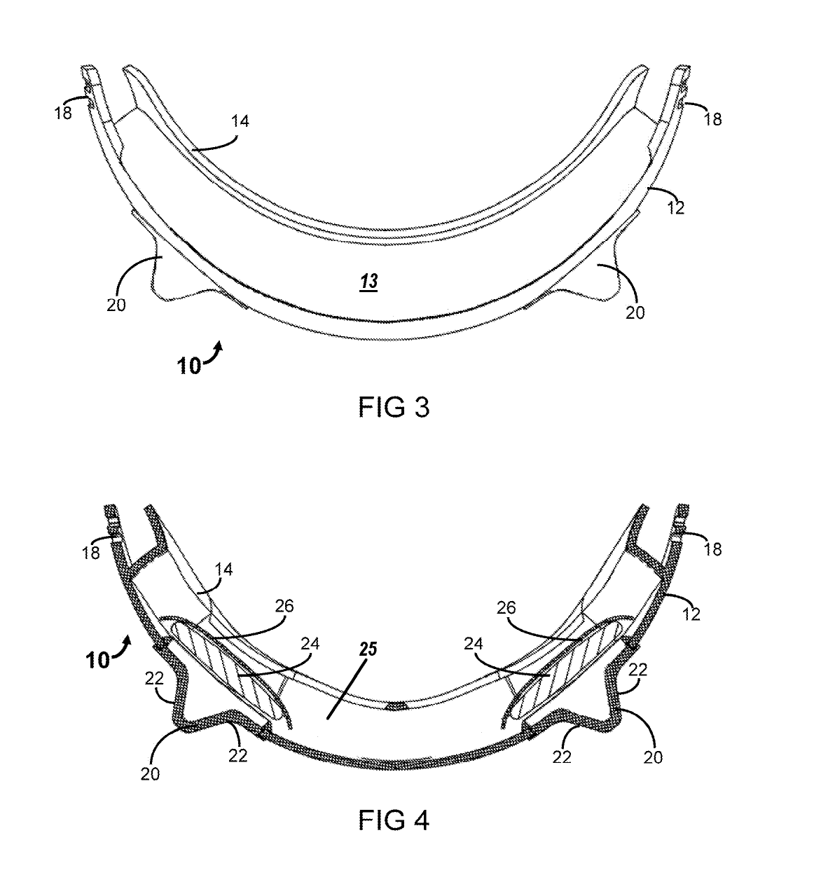

Referring now to the drawings of FIGS. 1-10 there are shown various aspects of the device 10 and method herein. Shown in FIG. 1 there can be seen a perspective view of the mask 12 of the device 10, as it would be held in a biased engagement to the face of a user to the as-worn position by a flexible strap 13.

As shown in the drawings the device 10 is formed to a mask 12 configuration, having an endwall 15 communicating to a traverse sidewall 13 which defines a permiter footprint on the face of the user which is of sufficient size to position a treatment cavity, 25 adjacent to and covering both eye sockets of the human wearer. The sidewall 13 is of a permiter shape and length to form an offset of the treatment cavity 25 which extends from 1 to 2 inches in a surrounding area of each eye socket. This surrounding portion of the treatment cavity 25 provides a means to communicate temperature and moisture therapy to the skin and tissue surrounding the eye to the distance of the surrounding area.

The mask endwall 15 and sidewall 13 are shaped about their respective perimeters to provide a U-shaped cutout 16 area, per FIGS. 1 and 2 which allows for comfortable communication of the user's nose therethrough. The sidewall 13 shown as edges of the mask endwall 15 of the mask sidewall 13, project in a direction traverse to the curved plane of the endwall 15 and toward the face of the user to form a seal therewith along the circumference of the endwall 15. The endwall 15 has a flexible edge 14 portion which is of a durometer to be soft and pliable and conform in shape to the face of the user to form a seal. The flexible edge 14 if part of the sidewall 13 may bend tangent to the facial anatomy at key locations to form means to increase the surface area of the contact of the edge of the sidewall 13 and the face, and an enhanced seal against the face.

Also shown in FIG. 1, the mask 12 provides means for cooperative engagement with the strap in the form of slots 18. The slot or slots 18 provide a pathway and thus are adapted for attachment to the elastic strap which may be employed with the device 10 to provide a means to bias the edge of the sidewall 13 to a seal pressed against the user's face. The slots 18 and strap can alternatively be replaced with hooks, velcro, buttons, snaps, or temples, or other fastening components to provide a means for engagement of the mask 12 to the as-worn position. The fastening feature must be designed for extended use and wear and to accommodate bands and straps of various sizes to accommodate various head sizes as can the size of the mask 12 by varying the size of the endwall 15 and extending sidewall 13 to accommodate different sized and shaped faces.

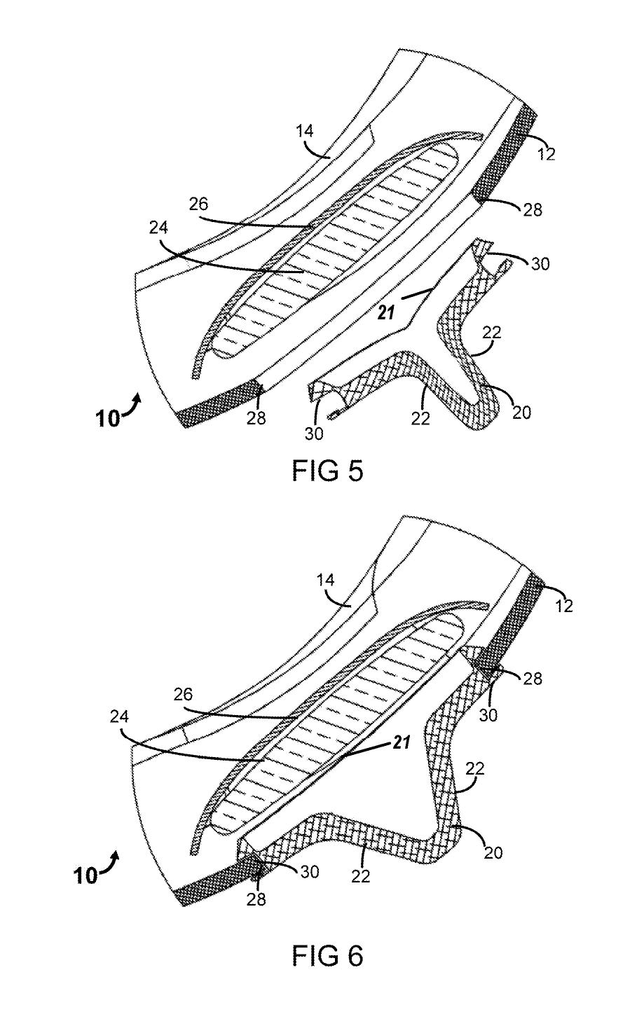

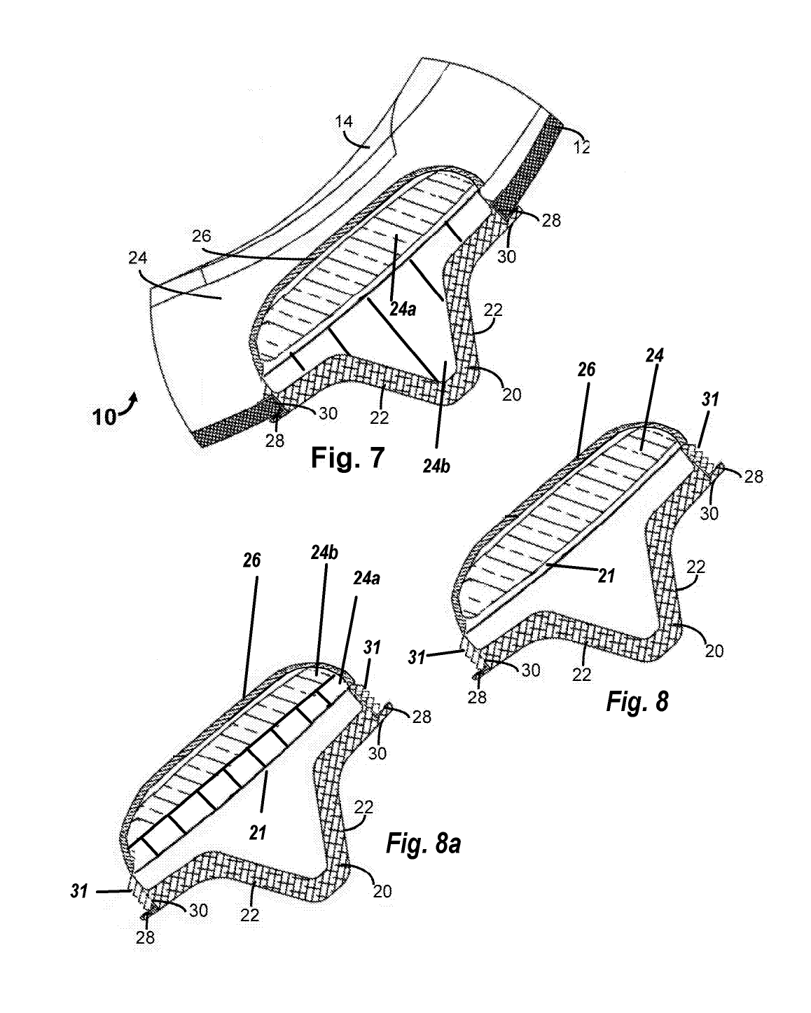

As depicted in FIG. 5-6, the mask 12 contains two apertures 28, preferably placed to align with the adjacent user's eyes or close thereto. Pads 24, which may be thermal reservoirs of heat or cold, such as gel packs, which may be heated or chilled, or which may be liquid reservoirs to communicate moisture to the treatment cavity 25, or combinations thereof, can be placed within the cavity 25. Placement may either be placed against a containment wall 26 as in FIG. 4-6, or may be engaged with the leading surface of the plug 20 as in FIGS. 7-10. The containment wall 26 is preferably a flexible textile material or sheet material formed to allow passage of water vapor therethrough such as a woven or non woven fabrics or polymer sheeting with perforations communicating between both sides of the sheeting.

These apertures 28 are also designed complimentary to the shape of each accept a sealing plug 20, to allow for a removable sealed engagement therein. This effectively allows the pads 24 and their contents of heat, cold, or moisture, or combinations thereof, to be in a sealed communication withing the treatment cavity 25, within the device 10. This allows for an ongoing communication of one or a combination of heat, cold, and moisture to the treatment cavity 25 which is engageable as well as renewable through the endwall 15 without the need to remove the mask 12 from the as-worn position.

When the pads 24 providing the thermal reservoir and/or moisture reservoir to supply the cavity 25 are separated from the plug 20, a sheet of perforated containment wall 26 is positioned within the cavity 25 adjacent a position to align with each eye when in the as-worn position. This maintains the pads 24 and their communicated heat, cold, and/or moisture, operatively positioned within the cavity 25 and immediately adjacent to, or if desired, in a biased contact with, the eyelid and/or eye socket area of the face of the wearer.

The contact pressure can be adjusted for more or less force, by employing an elastic fabric or sheet of perforated material for the containment wall 26, and forming the plugs 20 with a plug endwall 21 which projects a distance more, or less, toward the eye of the user when in sealed engagement with the apertures 28.

Alternatively, the circumference of the plug 20 at the engagement point with the aperture 28, may be threaded 31 or configured in some other fashion to translate in its engagement through the aperture 28 and provide a means to translate the plug a distance toward and away from the user's eye. Moving the plug endwall 21 toward the eye will increase pressure of the pad 24 by stretching the elastic sheet forming the containment wall 26, while rotating the plug 20 to move the plug end wall 21 away from the eye of the user will reduce or eliminate any pressure of the pad 24 on the eye.

The sealing plug 20, is preferably composed of a durable but flexible plastic or polymeric material which is substantially waterproof and will not pass air therethrough. Further the material should be of a type which can withstand direct contact with the heat or cold of pads 24 which are thermal reservoirs, and hold up to the moisture should the pads 24 be moisture pads 24. In the mode which is pinched for removal, the plug 20 has a circular engagement end and a V-shaped cross section with two angled V-walls 22 as in FIG. 5. Channels or threads around the sealing plug's 20 perimeter allow it to seal against the edge of the aperture 28 within the endwall 15 or sidewall 13 of the formed mask 12.

In the mode of the plug 20 which rotatably engages such as in FIG. 7-8, a compressive engagement may be achieved as in FIG. 7 by pressing the plug 20 which engaged to one or more pads 24, into the aperture 28. If translation is desired to adjust a pressure to the pad 24 where either a containment wall 26 holds the pad 24, or where the pad 24 is engaged to the end of the plug 20, using a containment wall 26 or other means, as in FIGS. 7-8, then threads 31 shown in FIG. 8 or other means for translatable engagement of the plug 20 through the aperture 28 may be employed.

One mode of the procedure or method for sealing the device 10 subsequent to a thermal and/or moisture pad 24 insertion, can be seen in FIG. 5-6, whereas, the user pinches the sealing plug's 20 V-walls 22 to compress it, and allow the sealing plug to insert and nest into the orifices 28 in the mask 12. The other mode as noted using the plug 20 of FIG. 8, may employ a rotation and threads 31 to translate the plug 20 into the aperture 28, and thereby also translate the plug endwall 21 toward and away from the user's eye.

The thermal reservoir 24a and/or moisture reservoir 24b formed as pads 24, as shown in the various figures, may be employed combined or separate and can be composed of glycerin beads, sodium acetate, clay, water, foam, fabric, rice or other materials which can hold in a dispensable reservoir, sufficient moisture to communicate to the cavity 25. Such materials are also easily be heated in a microwave or conventional oven, or cooled in a freezer or refrigerator to thereby provide a combination thermal and hydration reservoir to communicate such to the cavity.

The pads 24, when configured as thermal reservoirs 24a can also be engineered to dissipate heat over the defined period of time, and with measures to prevent overheating or burning the user. This is accomplished using a separate material as a thermal reservoir 24a for heat or cold such as a gel pak formed of a thermally conductive gel held withing a flexible housing. Pads 24 formed of such gel pak material can be combined with pads 24 formed of moisture reservoir 24b material acting as a hydration reservoir, may be used in combinations to achieve the heat and/or moisture desired in the treatment chamber 25 such as in FIGS. 8-10.

Further, the pads 24 formed as thermal reservoirs 24a and pads 24 providing a moisture reservoir 24b, can be disposable or reusable, and can be designed to be inserted into a sleeve made of gauze, fabric, foam or other materials to aid with moisture and heat dissipation. The mode of the plug 20 of FIG. 8a can be employed with any embodiment of the device and is especially well configured to place a thermal reservoir 24a section adjacent a pad 24 which is a moisture reservoir 24b, in a single unit which may hydrated and then heated or chilled and engaged in the aperture. Such combination plugs 20 can be provided in sterile wrapping and heated or chilled subsequent to unwrapping and then engaged.

FIG. 7 depicts a mode of the device of FIG. 8 engaged to the mask 12, wherein the pad 24 is a hydrating pad formed of a material adapted to be a moisture reservoir 24b and is placed adjacent a pad 24 configured as a thermal reservoir 24a which is formed by the gel pack which will communicate temperature change to the cavity 25 by providing thermal conduction and concurrently heating the hydrating pad 24 adjacent thereto to encourage heated moisture to the cavity 25.

As noted above FIG. 8 depicts the unitary mode of pad 24 and plug 20 which may either have a pad 24 formed of thermal reservoir 24a material, or a hydration reservoir 24b material, or a combination of both, all engaged to the plug 20 and easily inserted as a unit to the aperture 28 for communication to the cavity 25.

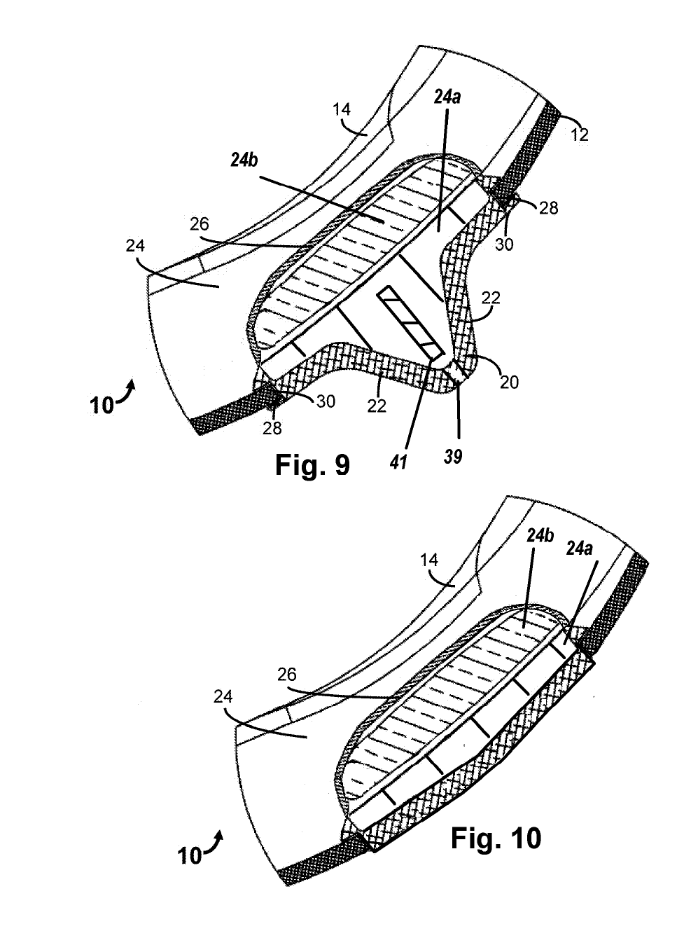

FIG. 9 show a mode of the plug 20 of any of the figures herein. In this mode the plug 20 has a slot 39 which is configured for insertion of a supplemental component 41 to add an additional reservoir and supply of heat, cooling, or hydration during a treatment without removing the plug 20 from the mask 12.

Finally, FIG. 10 depicts a mode of the device 10, with a plug 20 which inserts from the interior side of the endwall 15 within the treatment cavity 25 which may be desirable with some users.

While all of the fundamental characteristics and features of the device herein have been disclosed and described, with reference to particular embodiments thereof, a latitude of modification, various changes and substitutions are intended in the foregoing disclosure and it will be apparent that in some instance, some features of the invention will be employed without a corresponding use of other features without departing from the scope of the invention as set forth. It should be understood that such substitutions, modifications, and variations may be made by those skilled in the art without departing from the spirit or scope of the invention. Consequently, all such modifications and variations are included within the scope of the invention as defined herein.

What is claimed is:

1. An eyewear apparatus for maintaining a sealed cavity in front of a user's eye surrounding area when engaged to the face in an as-worn position, comprising:
a cupped semi-cylindrical mask which sufficiently covers the skin around both eyes, with an extended circumferential edge;
said mask having projecting edge portion formed of flexible material, extending from said circumferential edge, said projecting edge portion having a first surface positionable in contact with said face of said user in a seal, when in said as-worn position;
said sealed cavity defined by the area between an interior surface of said curved mask portion and said face of said user covering said eye surrounding area, when said mask is worn in said as-worn position;
said projecting edge deformable into said cavity by pressure thereon from outside said cavity;
said sealed cavity providing means to maintain higher humidity and temperature differential in said cavity

2. The eyewear apparatus of claim 1, additionally comprising:
said first surface positionable said projecting edge portion being substantially planar thereby providing means to increase a contact area of said seal.

3. The eyewear apparatus of claim 1, additionally comprising:
said curved lens portion having an exterior surface opposite said interior surface;

4. The eyewear apparatus of claim 1, additionally comprising:
means for biased engagement of said first surface of said projecting edge portion in contact with said face of said user.

5. The eyewear apparatus of claim 2, additionally comprising:
means for biased engagement of said first surface of said projecting edge portion in contact with said face of said user.

6. The eyewear apparatus of claim 3, additionally comprising:
means for biased engagement of said first surface of said projecting edge portion in contact with said face of said user.

7. The eyewear apparatus of claim 1, additionally comprising:
said projecting edge having a second surface, opposite said first surface;
said first surface formed of thermoplastic material which is softer than said second surface;
said first surface thereby providing means for cushioning for increased comfort of said user when said eyewear apparatus is in said as-worn position for long time periods.

8. The eyewear apparatus of claim 1, additionally comprising:
two orifices and containing walls within said mask to accept and contain thermal and moisture pads;

9. The eyewear apparatus of claim 2, additionally comprising:
two orifices and containing walls within said mask to accept and contain thermal and moisture pads.

10. The eyewear apparatus of claim 1, additionally comprising:
two plugs designed to house within said orifices within said mask to confine temperature and moisture within the mask.

11. The eyewear apparatus of claim 2, additionally comprising:
two plugs designed to house within said orifices within said mask to confine temperature and moisture within the mask.
